(19) United States
(12) Patent Application Publication
(10) Pub. No.: US 2026/0240481 A1
(43) Pub. Date: Aug. 20, 2026

(54) Implantable Medical Device for Sensing Electrocardiogram Signals

(71) Applicant: BIOTRONIK SE & Co. KG, Berlin (DE)

(72) Inventors: R. Hollis WHITTINGTON, Portland, OR (US); Dirk MUESSIG, West Linn, OR (US); Volker LANG, Berlin (DE); Andreas BECKER, Tigard, OR (US)

(73) Assignee: BIOTRONIK SE & Co. KG, Berlin (DE)

(21) Appl. No.: 19/469,303

(22) PCT Filed: Mar. 11, 2024
(86) PCT No.: PCT/EP2024/056383

§ 371 Date: Sep. 25, 2025

Related U.S. Application Data
(60) Provisional application No. 63/456,835, filed on Apr. 4, 2023.

(30) Foreign Application Priority Data
Apr. 19, 2023 (EP) 23168649
Apr. 19, 2023 (EP) 23168649.4

(51) Int. Cl. A61B 5/346; A61B 5/00; A61B 5/29

(52) U.S. Cl. CPC A61B 5/346; A61B 5/29; A61B 5/686; A61B 2560/0204

(57) ABSTRACT

An implantable medical device for sensing electrocardiogram signals includes an arrangement of electrode poles configured to sense electrocardiogram signals, and a processing module for processing electrocardiogram signals received via said arrangement of electrode poles. The processing module is configured to switch between a first mode of operation and a second mode of operation. The processing module, in said first mode of operation, is configured to identify a predefined event based on sensed electrocardiogram signals and to record a snapshot signal representation of electrocardiogram signals over a first duration of time. The processing module, in said second mode of operation, further is configured to record a continuous signal representation of electrocardiogram signals over a second duration of time larger than said first duration of time.

### CROSS-REFERENCE TO RELATED APPLICATIONS

This application is the United States National Phase under 35 U.S.C. § 371 of PCT International Patent Application No. PCT/EP2024/056383, filed on Mar. 11, 2024, which claims the benefit of European Patent Application No. 23168649.4, filed on Apr. 19, 2023, and U.S. Provisional Patent Application No. 63/456,835, filed on Apr. 4, 2023, the disclosures of which are hereby incorporated by reference herein in their entireties.

### TECHNICAL FIELD

The present invention generally relates to an implantable medical device for sensing electrocardiogram signals which is to be implanted into a patient for carrying out a diagnostic and/or therapeutic function.

### BACKGROUND

An implantable medical device of this kind may for example be a pacemaker, an implantable cardioverter defibrillator, a sensor device such as a bio-sensor, or a monitoring device. The implantable medical device herein is configured to sense electrocardiogram signals.

For example, the implantable medical device may be a monitoring device which is configured to record electrocardiogram signals and to communicate recorded electrocardiogram signals or information derived from recorded electrocardiogram signals to an external device in the context of a remote monitoring system.

An implantable medical device as e.g. used in a remote monitoring system shall allow for a reliable monitoring of a physiological state of a patient. In particular, using the implantable medical device it shall be possible to reliably detect an abnormal cardiac state based on recorded electrocardiogram signals. If an abnormality is detected in electrocardiogram signals, the implantable medical device shall be enabled to communicate with for example an external device of a remote monitoring system in order to trigger a message to a service center to alert medical personnel of a potential need for attention.

An implantable medical device, for example a monitoring device, of the type concerned herein generally is configured for implantation in a patient. For recording electrocardiogram signals the implantable medical device comprises an arrangement of electrode poles configured to sense electrocardiogram signals. A processing module is configured for processing electrocardiogram signals received via said arrangement of electrode poles.

In a conventional implantable medical device such as a monitoring device, a recording of signals generally takes place in an event-driven manner. If a certain event, for example relating to a cardiac arrhythmia, such as an atrial or ventricular fibrillation, is identified based on sensed signals, a signal snapshot is recorded indicative of the particular, identified event, and the signal snapshot is communicated to an external device, for example within the context of a remote monitoring system, in order to enable an analysis of the signal snapshot by a physician.

Whereas implantable medical devices, such as implantable monitoring devices, are implanted in a patient and operate for sensing and recording signals while resting within the patient, other monitoring devices, such as so-called Holter recorders, are configured for use outside of the patient. Such external recording devices typically use an arrangement of ECG leads having electrodes to be placed on the patient's skin such that electrocardiogram signals may be recorded externally to the patient. Typically herein, a recording may take place over a prolonged duration of time, for example a time span between 24 hours and 30 days, in order to allow a physician to analyze electrocardiogram signals for example with respect to the occurrence of infrequent arrhythmias.

U.S. Pat. No. 10,827,929 describes systems and methods for obtaining high-resolution data from implantable medical devices by triggering a system behavior change. For example, data may be batched and communicated to an external device, the external device being configured for reconstructing the batched data.

The present disclosure is directed toward overcoming one or more of the above-mentioned problems, though not necessarily limited to embodiments that do.

### SUMMARY

It is an object of the present invention to provide an implantable medical device and a method for operating an implantable medical device which in a reliable way allow to monitor electrocardiogram signals in varying scenarios.

This object is achieved by means of an implantable medical device comprising the features of claim 1.

In one aspect, an implantable medical device for sensing electrocardiogram signals comprises an arrangement of electrode poles configured to sense electrocardiogram signals and a processing module for processing electrocardiogram signals received via said arrangement of electrode poles. The processing module is configured to switch between a first mode of operation and a second mode of operation. The processing module, in said first mode of operation, is configured to identify a predefined event based on sensed electrocardiogram signals and to record a snapshot signal representation of the electrocardiogram signals over a first duration of time. The processing module furthermore, in said second mode of operation, is configured to record a continuous signal representation of electrocardiogram signals over a second duration of time larger than the first duration of time.

For example, the implantable medical device may be a monitoring device. In another embodiment, the implantable medical device is a cardiac stimulation device, such as a cardiac pacemaker device or a cardiac defibrillation device, for example an implantable cardioverter defibrillator. The implantable medical device may be configured for implantation external to the patient's heart, e.g. for subcutaneous implantation. In another embodiment, the implantable medical device may be configured for implantation partially or wholly within the patient's heart.

The implantable medical device may be a leadless device, or may comprise one or multiple leads carrying one or multiple electrode poles.

The implantable medical device is configured to sense electrocardiogram signals making use of an arrangement of electrode poles, for example on a housing of the implantable medical device or on one or multiple leads extending from a housing of the implantable medical device. Electrocardiogram signals are processed by means of a processing module in order to enable a recording of data relating to electrocardiogram signals, such that for example data may be communicated to an external device in order to enable e.g. a monitoring within the context of a remote monitoring system.

Herein, the processing module is configured to operate in different modes of operation, wherein the processing module may switch from one mode of operation to another and vice versa.

Specifically, in a first mode of operation the processing module is configured to identify a predefined event based on sensed electrocardiogram signals and to record a snapshot signal representation of electrocardiogram signals over a first duration of time. In the first mode of operation the processing module generally processes signals as sensed by means of the arrangement of electrode poles, wherein a recording of signals however takes place in an event-driven manner in order to record a snapshot signal representation indicative of the particular identified (predefined) event. If for example a particular arrhythmia event is identified, a snapshot signal representation spanning over one or multiple cardiac cycles is recorded, the snapshot signal representation reflecting the particular (predefined) event and allowing to assess the (predefined) event upon communicating the snapshot signal representation to an external device.

In the following, the shorter term ‘snapshot’ is also used for the term ‘snapshot signal representation’.

In the first mode of operation, hence, sensed signals are not recorded and stored continuously over a prolonged period of time, but in an event-driven manner. If a particular, predefined event, such as an arrhythmia event, for example relating to a bradycardia or a tachycardia, is identified based on sensed electrocardiogram signals, the snapshot is recorded and stored for communication to an external device.

In addition, the processing module is configured to operate in a second mode of operation in which a recording does not take place in an event-driven manner with relation to a particular (predefined) event, but in a continuous manner. In the second mode of operation a continuous signal representation is recorded over a (second) duration of time which is larger than the (first) duration of time over which the snapshot signal representation is recorded during the first mode of operation. In the second mode of operation, hence, a recording of data takes place similar to a so-called Holter recorder, allowing the recording of signals over a prolonged period of time and hence allowing for an assessment of a patient's cardiac behavior over the entire continuous period of time spanned by the recorded data.

Within the implantable medical device, hence, an event-driven snapshot recording is combined with the capability of a continuous recording over a prolonged period of time. The processing module may switch between one mode of operation and the other such that, based on a user configuration or based on certain scenarios as identified by the processing module, one mode of operation or the other may be used for recording data.

In one embodiment, the first duration of time lies in a range between 1 second and 5 minutes. In particular, the first duration of time may extend over a time span allowing for analyzing a particular (predefined) event, such as an arrhythmia event.

The first duration of time may be set to assume a particular time value. In another embodiment, the first duration of time may be set to span a predefined number of cardiac cycles.

In one embodiment, the second duration of time lies in a range between 1 hour and 50 days. The second duration of time generally is larger than the first duration of time. Within the second duration of time a continuous recording of data, in particular relating to sensed electrocardiogram signals, takes place. The second duration of time may for example lie in a range between 1 day and 30 days, for example 1 day (24 hours), 2 days (48 hours), 5 days, 7 days, 10 days days 20 days, 25 days or 30 days, and generally extends over a prolonged time span over which electrocardiogram signals are continuously recorded. i.e. at a discrete sampling rate and with a defined resolution.

In one embodiment, the processing module is configured to store, in the first mode of operation, data indicative of the snapshot signal representation at a first sampling rate and/or a first compression rate and, in the second mode of operation, data indicative of the continuous signal representation at a second sampling rate smaller than the first sampling rate and/or a second compression rate larger than the first compression rate. Generally, as data is recorded in the second mode of operation over a prolonged time span, the amount of data recorded is large in the second mode of operation. In order to be able to store the recorded data in the device memory, which generally has a limited size in an implantable medical device, the sampling rate in the second mode of operation may be reduced in comparison to the recording of data in the first mode of operation. Alternatively or in addition, data may be stored in the second mode of operation by employing an increased compression such that data is stored efficiently at a reduced size in the second mode of operation.

For example, in the first mode of operation a resolution of 16 bit may be employed. In turn, in the second mode of operation a reduced resolution of for example 8 bit may be employed, such that the resolution is reduced in the second mode of operation.

Alternatively or in addition, filtering conditions, such as a high-pass bound, may be adapted in the second mode of operation in comparison to the first mode of operation. For example, in the first mode of operation a larger bandwidth for recording data then in the second mode of operation may be employed or vice versa.

Alternatively or in addition, the sampling rate of the signal may be adapted in the second mode of operation in comparison to the first mode of operation. For examples, in the first mode of operation a higher sampling rate may be used such as equal to or larger than 250 samples per second, whereas the sample rate of the second mode might be smaller than 250 samples per second in order to allow a longer time duration of recording in the second mode with the available storage of the device. Other cutoffs may be appropriate. The first mode may also use a lower sample rate whereas the second mode uses a higher sample rate.

In one embodiment, the processing module is configured to store, in the second mode of operation, data relating to or originating from at least one further sensor signal obtained using a sensor different than the arrangement of electrode poles. Together with electrocardiogram signals, hence, further data stemming from other sensors may be stored. For example, data relating to physical activity, the patient's posture, respiratory parameters such as the respiration rate or minute ventilation, heart sounds, oxygen saturation, and a thoracic impedance may be stored together with electrocardiogram signals. For this, a motion sensor, a flow sensor, an impedance sensor, an oxygen sensor or the like may be employed in order to derive information relating to further parameters which are then recorded and stored together with electrocardiogram signal information.

In one embodiment, the processing module is configured to identify, in the second mode of operation, an occurrence of a predefined event during the recording of the continuous signal representation and to store a marker indicative of the occurrence of the predefined event together with the continuous signal representation. Generally, during the second mode of operation, a signal processing may be reduced in that signals may be recorded without a content-based processing, in particular without employing a pattern recognition or the like in order to identify certain (predefined) events. However, in addition to the continuous recording of data in the second mode of operation, an analysis may take place in which certain (predefined) events are identified in order to derive markers indicative of the (predefined) events. For example, arrhythmia events, such as events relating to bradycardia, tachycardia, asystole, sudden rate changes, atrial fibrillation, atrial flutter, atrial tachycardia, ventricular tachycardia or the like, may be identified, and corresponding markers may be stored together with the continuous signal representation. Hence, in addition to the continuous signal representation, markers are recorded, indicating at what times during the continuous signal recording certain (predefined) events have occurred. This allows, when communicating the recorded signal representation to an external device, for an easy assessment of the recorded signal in that a further processing of the signal may not be necessary, but has been carried out already by the implantable medical device. A physician who analyzes the recorded signals hence is immediately notified of the occurrence of certain (predefined) events during the time span of recording, such that analysis and diagnosis becomes easy and efficient.

Because the implantable medical device may identify (predefined) events with an accuracy as common in current implantable medical monitoring devices, no further offline post-processing of recorded signals as obtained in the second mode of operation may be required. Rather, in the second mode of operation events may be identified and classified and may be indicated, with reference to the specific time of occurrence, in the continuously recorded data such that a physician immediately is notified about the occurrence of certain (predefined) events within the recorded data as obtained in the second mode of operation.

In one embodiment, the implantable medical device comprises a communication circuitry for communicating, during or subsequent to execution of the first mode of operation, data relating to the snapshot signal representation and, during or subsequent to execution of the second mode of operation, data relating to the continuous signal representation to an external device. Generally, during the first mode of operation data relating to the snapshot signal representation is recorded and stored in the device memory, upon which the data may be communicated to the external device together with information about the (predefined) event which has triggered the snapshot recording. Likewise, in the second mode of operation data relating to the continuous signal representation is stored in the device memory. It herein is conceivable that the data throughout the entire duration of time of operation in the second mode of operation is stored in the device memory and is communicated to the external device only upon termination of the second mode of operation, that is upon lapse of the second duration of time. In another embodiment, certain portions of data may be repeatedly transmitted to the external device during execution of the second mode of operation, such that repeatedly after for example a predefined time period data is transmitted to the external device. The repeated transmission of portions of the data may ease the requirements for the device memory, in that not the full amount of data during the entire (second) duration of time of recording in the second mode of operation needs to be stored, but only a portion which, after transmission to the external device, may be overwritten.

The external device generally rests outside of the patient. The external device for example may be an external communication device, such as a smart phone or a tablet computer. In another embodiment, the external device may be a patient device in the context of a remote monitoring system. The external device may be worn by the patient on the body, or, in another embodiment, is not continuously worn by the patient, but for example is (sporadically) handheld or stationary in the remote of the patient.

The external device, in one embodiment, is configured, within a system comprising the implantable medical device and the external device, to store the data received from the implantable medical device. In one embodiment, the external device is configured to transmit the data to a server, such as a remote monitoring service center.

The transmission between the implantable medical device and the external device may employ common communication schemes and protocols. For example, data transmission may be encrypted.

In order to reduce the battery load of the implantable medical device, the implantable medical device may employ low-power techniques, including for example a duty cycling scheme for transmission of data to the external device at a given duty cycle load. Alternatively or in addition, the implantable medical device may employ low-power analog and/or digital electronics in order to minimize standby, transient, and operating currents. Yet alternatively or in addition, the implantable medical device may employ low-power data transmission techniques such as Bluetooth Low Energy (BLE), direct electric field communication to an external device or other implanted devices, magnetic field communication, or ultrasound communication. A nearfield detection may be employed, for example along with a proximity indicator, to transmit data to the external device only when it is recognized that the external device is nearby and hence available for a signal communication, thus reducing the transmit and receive power required by the implantable medical device.

In one embodiment, the implantable medical device comprises a battery module, in particular a rechargeable battery module. In particular when the implantable medical device is configured for subcutaneous implantation, the recharging of the battery module may take place in an implanted state of the implantable medical device, for example inductively using an external charging device placed in the vicinity of the implantable medical device.

In one embodiment, the processing module is configured, in said first mode of operation, to record said snapshot signal representation according to user configurable first settings, said user configurable first settings including at least one of said first duration of time, a selection of detected parameters, a use of one of a multiplicity of available event detection algorithms, a power consumption setting, a sampling rate setting, a compression rate setting, and a communication setting.

In one embodiment, the processing module is configured, in the second mode of operation, to record the continuous signal representation according to user configurable second settings. The user configurable second settings include at least one of the second duration of time, a selection of detection parameters, the use of (at least) one of a multiplicity of available event detection algorithms, a power consumption setting, a sampling rate setting, a compression rate setting, and a communication setting.

Hence, operation of the implantable medical device in the first and/or in the second mode of operation may be user configurable. The user, prior to implantation or in an implanted state of the implantable medical device, may adjust the settings of the implantable medical device, such that operation of the implantable medical device in the first mode and/or in the second mode of operation takes place according to a particular programming by the user.

In the following, the shorter term ‘duration’is also used for the term ‘duration of time’.

In particular, the duration over which data is recorded in the second mode of operation may be user configurable, such that a user may select and specify the particular duration of recording.

Alternatively or in addition, a user may select which parameters, together with electrocardiogram signals, are recorded, such as parameters relating to physical activity, posture, respiratory parameters or the like. Hence, a user may select what kind of data together with electrocardiogram signals is recorded during the second mode of operation.

Yet alternatively or in addition, a user may select which particular algorithm shall be employed during the second mode of operation to detect certain (predefined) events in order to derive corresponding markers. For example, a user may select whether (predefined) events relating to bradycardia, asystole, atrial fibrillation, atrial tachycardia, ventricular tachycardia or another condition shall be detected and recorded throughout the second mode of operation.

The user may adjust a power consumption setting, a sampling rate and/or a compression rate to be employed during the second mode of operation.

The user may also configure whether data shall be communicated only after termination of the second mode of operation, or whether portions of data shall be communicated to an external device already during execution of the second mode of operation.

Alternatively or in addition to a user configuration capability, the processing module may be configured, prior to or during the second mode of operation, to automatically adapt settings for recording the continuous signal representation. The automatically adaptable settings may for example include at least one of the second duration of time, the selection of detected parameters, the use of (at least) one of a multiplicity of available event detection algorithms, a power consumption setting, a sampling rate setting, a compression rate setting, and a communication setting, as specified above.

In one embodiment, the processing module is configured to initiate the second mode of operation based on a detection of at least one (predefined) event. Generally, the second mode of operation may be initiated by a user, which may for example, by using e.g. an external programmer or an external communication device or by remotely using a remote server, cause the implantable medical device to trigger the second mode of operation and hence to start a continuous recording of data. Alternatively or in addition, the processing module may be configured to automatically start the second mode of operation on the occurrence of a particular (predefined) event or a sequence of (predefined) events. The processing module may for example continuously process signals in order to identify, based on sensed electrocardiogram signals, whether a particular condition, such as an arrhythmia condition, for example a bradycardia or a tachycardia, exists and develops in a certain way. If it for example is identified that a certain heart condition develops over time, the processing module may automatically start the second mode of operation in order to continuously record data in the second mode of operation.

For example, if detected (predefined) events indicate that the patient slowly develops apneas over time, the processing module may start the second mode of operation in order to continuously record data to allow a thorough assessment of a cardiac condition, such as a developing heart failure.

The processing module, for identifying a trigger condition in order to trigger the second mode of operation, may for example employ an algorithm, for example an algorithm including an artificial intelligence (AI) scheme. The algorithm may in particular take into account patient properties, past arrhythmia properties, and/or other input parameters to determine when it is appropriate to trigger the second mode of operation.

The algorithm may also automatically adapt parameters to be used during the second mode of operation, such as a sample rate, a compression parameter or the like.

The algorithm, for example including artificial intelligence, may also command the communication circuitry to connect and communicate data to the external device in order to optimize data transmission and maximize coverage, and at the same time optimize power consumption by the communication circuitry.

The implantable medical device may, in one embodiment, comprise two or more electrode poles, e.g. arranged on a generator device housing and/or on one or multiple electrode leads extending from a generator device. By means of the different electrode poles an electrical coupling to surrounding tissue is established when the implantable medical device is implanted in a patient, such that electrocardiogram signals may be sensed using the different electrode poles.

Electrocardiogram signals herein may be sensed using one or more pairs of electrode poles to obtain different electrocardiogram signals, which may be processed by the processing module in order to assess a cardiac function. If an abnormality based on a monitoring of a particular cardiac function is detected in the first mode of operation, this may be interpreted as a change in a physiological state of the patient, and accordingly a snapshot signal representation relating to this event may recorded and for example communicated to an external device to trigger an alert in a remote monitoring system.

In another aspect, a method for operating an implantable medical device for sensing electrocardiogram signals comprises: sensing electrocardiogram signals using an arrangement of electrode poles of the implantable medical device; processing, using a processing module of the implantable medical device, electrocardiogram signals received via said arrangement of electrode poles; identifying, by the processing module in a first mode of operation, a predefined event based on sensed electrocardiogram signals and recording a snapshot signal representation of electrocardiogram signals over a first duration of time; and recording, by the processing module in a second mode of operation, a continuous signal representation of electrocardiogram signals over a second duration of time larger than said first duration of time.

The advantages and advantageous embodiments described above for the implantable medical device equally apply also to the method, such that it shall be referred to the above in this respect.

When in the above it is referred to a signal representation, in particular a snapshot signal representation or a continuous signal representation, this shall relate to recorded (single channel or multi-channel) electrocardiogram signals, wherein the signal representation may be the raw data of the electrocardiogram signals as sensed by the electrode pole arrangement or may be a processed version of the electrocardiogram signals, such as a compressed version of the electrocardiogram signals. A signal representation hence represents sensed electrocardiogram signals, which in the first mode of operation are recorded to obtain a snapshot relating to an identified (predefined) event and in the second mode of operation are recorded over a prolonged time span in order to obtain a continuous recording of electrocardiogram signals.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein,

FIG. 1 shows a schematic drawing of an implantable medical device implanted in a patient;

FIG. 2 shows a schematic drawing of an embodiment of an implantable medical device comprising an arrangement of electrode poles; and

FIG. 3 shows a schematic drawing of a processing module configured to operate in different modes of operation to record electrocardiogram signals in an event-driven manner or in a continuous manner.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

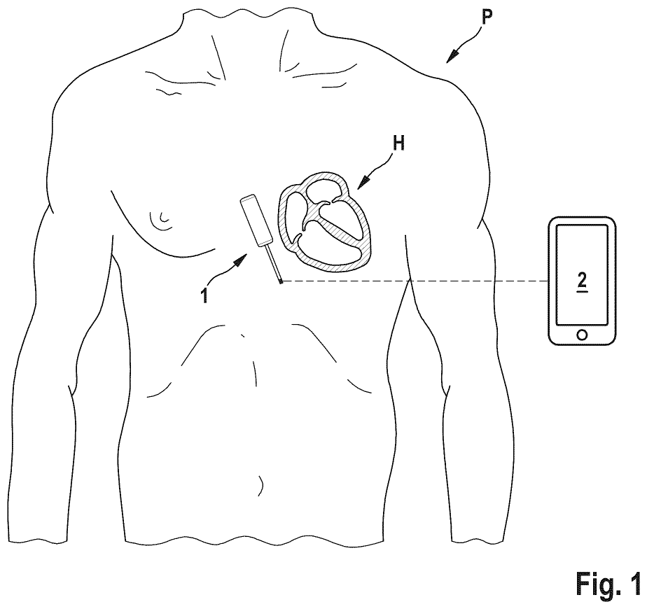

Referring to FIG. 1, in one embodiment an implantable medical device 1 is implanted (for example subcutaneously) into a patient P for serving a therapeutic and/or diagnostic function. The implantable medical device 1 may for example be implanted subcutaneously into the patient P for monitoring cardiac activity of the patient's heart H. The implantable medical device 1, for this, comprises an arrangement of electrode poles which are used to couple to surrounding tissue and to sense electrocardiogram signals originating from the heart H.

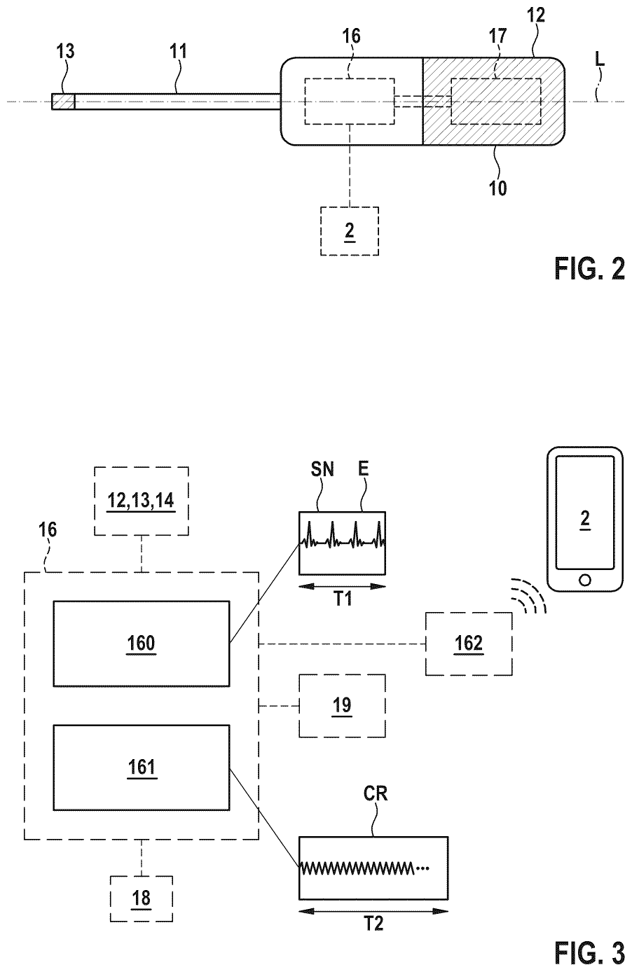

Referring now to FIG. 2, in one embodiment the implantable medical device 1 comprises a housing 10 formed e.g. by different housing segments, the housing 10 enclosing and encapsulating a processing module 16 (formed by electronic circuitry) and a battery module 17. In particular, a first housing segment may receive and enclose the battery module 17, or the first housing segment forms a housing of the battery segment 17 itself, whereas a second housing segment receives and encloses the processing module 16. A third housing segment longitudinally extends from the first and second housing segments and forms a lead portion 11 fixedly attached to the housing 10, having reduced cross-sectional dimensions with respect to the other (first and second) housing segments. The lead portion may be flexible and may for example mainly comprise electrically non-conductive materials.

In the embodiment of FIG. 2, a first electrode pole 12 is formed by the first housing segment enclosing the battery module 17, a second electrode pole 13 is arranged at a far end of the lead portion 11. The implantable medical device 1 with its housing 10 generally extends along a longitudinal axis L, the electrode poles 12, 13 being aligned along the longitudinal axis L and being axially displaced with respect to one another along the longitudinal axis L.

In the embodiment of FIG. 2 concerning a so-called leadless bio-monitor, the electrode poles 12, 13 may be formed by portions of the housing 10 itself, the housing 10 being made for example from an electrically conductive material, in particular a metal material. By exposing portions of the housing 10 towards the outside, the electrode poles 12, 13 are formed and may electrically contact with surrounding tissue in order to establish a coupling between the electrode poles 12, 13 to the surrounding tissue.

Using the arrangement of electrode poles 12, 13, electrocardiogram signals may be received and processed by the processing module 16. Based on the processing, a communication with an external device 2 may be established, for example to transmit alert messages to the external device 2 for example within the context of a remote monitoring system for monitoring a physiological state of the patient P.

Referring now to FIG. 3, the processing module 16 generally is configured to operate in different modes of operation to record data relating to electrocardiogram signals as sensed using the electrode arrangement comprising the electrode poles 12, 13. Herein, during the recording data may be sensed and may be processed, such that a recording of data relating to electrocardiogram signals may take place in either of the modes of operation of the processing module 16.

In the embodiment of FIG. 3, the processing module 16 is configured to operate in a first mode of operation 160 to record snapshot signal representations SN relating to particular identified (predefined) events E.

In the following, the shorter term ‘snapshot SN’ is also used for the term ‘snapshot signal representation SN’.

Generally, based on sensed electrocardiogram signals, the processing module 16 is configured to identify a particular (predefined) event E, such as a cardiac arrhythmia event, for example a bradycardia or a tachycardia, and following an event detection a snapshot SN of data is recorded over a specified duration of time T1 or over a specified number of cardiac cycles. The snapshot SN hence represents an electrocardiogram signal indicative of the particular identified (predefined) event E. The snapshot data is stored by the processing module 16, for example in a device memory 19, and may for example be communicated to an external device 2 by employing a communication circuitry 162.

In addition, the processing module 16 is configured to operate in a second mode of operation 161 in which data relating to electrocardiogram signals are recorded continuously over a prolonged period of time. In the second mode of operation, a continuous data recording over a substantially longer time period hence takes place, for example over a time period ranging from one hour to 50 consecutive days, for example between one day and 30 days.

In the second mode of operation 161, a continuous signal representation CR representing electrocardiogram signals over a duration of time T2 is obtained and is e.g. stored in the device memory 19.

Herein, during the second mode of operation 161, the complete continuous signal representation CR may be stored in the device memory 19, and may be communicated to the external device 2 using the communication circuitry 162 only upon termination of the second mode of operation. Alternatively or in addition, portions of the recorded data may be stored in the device memory 19 and, already during the execution of the second mode of operation 161, may be repeatedly communicated to the external device 2 using the communication circuitry 162, such that it is not necessary to store the complete amount of data relating to the continuous signal representation CR in the device memory 19 during the second mode of operation.

During the second mode of operation 161, a processing of data may be minimized. Herein, a sampling rate may be reduced in comparison to a sampling rate used during the first mode of operation 160. Alternatively or in addition, an increased compression may be employed in the second mode of operation 161 in order to reduce the amount of data to be recorded during the second mode of operation 161.

During the second mode of operation 161, in addition to the continuous recording of electrocardiogram signals, in one embodiment (predefined) events E may be identified based on sensed electrocardiogram signals, and markers indicative of the occurrence of certain (predefined) events E, such as bradycardia or tachycardia events, may be stored together with the continuous signal representation CR. Hence, in this embodiment a content-based processing of data takes place during the second mode of operation 161, and markers derived during the processing are stored and communicated together with the recorded electrocardiogram signals. Thus, a post-processing of the recorded data by the external device 2 or another system may be eased or entirely dispensable.

Alternatively, or in addition, in the second mode of operation 161 the (predefined) (snapshot) event E may be transmitted daily or like in the first mode of operation 160. Furthermore, continuous recording and events E may be sent out along the way.

In one embodiment, additional parameters may be recorded and stored together with the continuous signal representation CR. For example, the processing module 16 may receive sensor data from an additional sensor 18 (different than the arrangement of electrode poles 12, 13), such as a motion sensor, a pressure sensor, a flow sensor or another sensor. For example, information relating to physical activity of the patient, a posture, respiratory parameters such as a respiration rate or minute ventilation, heart sounds, oxygen saturation, and thoracic impedance may be recorded and stored together with the electrocardiogram signals.

The implantable medical device 1 may for example in a default state be configured to operate in the first mode of operation 160, such that the implanted medical device 1, for example an implantable monitoring device, in the default state operates according to the first mode of operation 160. The second mode of operation 161 may for example be triggered manually by a user, such as a physician, for example by employing an external programming device or a general external communication device, such as a smart phone or the like. The second mode of operation 161 hence is triggered upon a particular command issued by a user.

Alternatively or in addition, the implantable medical device 1 may be configured to automatically trigger the second mode of operation 161, for example upon the processing of signals and upon the occurrence of a particular sequence of (predefined) events E. For example, if a certain (predefined) event E, for example an arrhythmia event, or a particular development of (predefined) events E is identified, the second mode of operation 161 is triggered.

An algorithm employed by the processing module 16 for triggering the second mode of operation 161 may for example use artificial intelligence, such that the algorithm may adapt itself based on usage data and based on a continuous analysis of sensed (predefined) events E.

Settings to be employed during the first mode of operation 160 or the second mode of operation 161 may be user configurable, or may be automatically adapted by the processing module 16, for example by involving an artificial intelligence scheme. Such settings may for example relate to the duration of time for recording during the second mode of operation 161, to parameters to be recorded during the second mode of operation 161, to (predefined) events E which shall be identified and recorded during the second mode of operation 161, to a sampling rate setting, to a compression setting and/or to a power consumption setting, or to other parameters.

The implantable medical device 1, with its communication circuitry 162, may for example employ Bluetooth Low Energy (BLE), a direct electric field communication method, a magnetic field communication, or an ultrasound communication to communicate with the external device 2. In addition, a nearfield detection may be employed in order to identify whether the external device 2 is in proximity to the patient such that a data communication is enabled.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Housing
11 Header portion
12, 13 Electrode pole
16 Processing module
160, 161 Mode of operation
162 Communication circuitry
17 Battery module
18 Sensor
19 Device memory
2 External device
CR Continuous signal representation
E (predefined) Event
H Heart
L Longitudinal axis
P Patient
SN Snapshot signal representation
T1, T2 Duration of time

1. An implantable medical device for sensing electrocardiogram signals, comprising:
an arrangement of electrode poles configured to sense electrocardiogram signals; and
a processing module for processing electrocardiogram signals received via said arrangement of electrode poles;
wherein the processing module is configured to switch between a first mode of operation (160) and a second mode of operation;
wherein the processing module, in said first mode of operation, is configured to identify a predefined event based on sensed electrocardiogram signals and to record a snapshot signal representation of electrocardiogram signals over a first duration of time; and
wherein the processing module, in said second mode of operation, is configured to record a continuous signal representation of electrocardiogram signals over a second duration of time larger than said first duration of time.

2. The implantable medical device according to claim 1, wherein said first duration of time is in a range between 1 second and 5 minutes.

3. The implantable medical device according to claim 1, wherein said second duration of time is in a range between 1 hour and 50 days, preferably 1 to 7 days, more preferred 1 to two days.

4. The implantable medical device according to claim 1, wherein the processing module is configured to store, in said first mode of operation, data indicative of said snapshot signal representation at a first sampling rate and/or a first compression rate and, in said second mode of operation, data indicative of said continuous signal representation at a second sampling rate smaller than said first sampling rate and/or a second compression rate larger than said first compression rate.

5. The implantable medical device according to claim 1, wherein said processing module is configured to store, in said first and second mode of operation, data relating to or originating from at least one further sensor signal obtained using a sensor different than said arrangement of electrode poles.

6. The implantable medical device according to claim 1, wherein said the processing module is configured to identify, in said first or second mode of operation, an occurrence of a predefined event during the recording of said snapshot signal representation or continuous signal representation and to store a marker indicative of said occurrence of said predefined event together with said snapshot signal representation or continuous signal representation.

7. The implantable medical device according to claim 1, comprising a communication circuitry for communicating, during or subsequent to execution of said first mode of operation, data relating to said snapshot signal representation and, during or subsequent to execution of said second mode of operation, data relating to said continuous signal representation to an external device.

8. The implantable medical device according to claim 1, comprising a rechargeable battery module.

9. The implantable medical device according to claim 1, wherein the processing module is configured, in said first mode of operation, to record said snapshot signal representation according to user configurable first settings, said user configurable first settings including at least one of said first duration of time, a selection of detected parameters, a use of one of a multiplicity of available event detection algorithms, a power consumption setting, a sampling rate setting, a compression rate setting, and a communication setting.

10. The implantable medical device according to claim 1, wherein the processing module is configured, in said second mode of operation, to record said continuous signal representation according to user configurable second settings, said user configurable second settings including at least one of said second duration of time, a selection of detected parameters, a use of one of a multiplicity of available event detection algorithms, a power consumption setting, a sampling rate setting, a compression rate setting, and a communication setting.

11. The implantable medical device according to claim 1, wherein the processing module is configured, prior to or during said second mode of operation, to automatically adapt settings for recording said continuous signal representation, said settings including at least one of said second duration of time, a selection of detected parameters, a use of one of a multiplicity of available event detection algorithms, a power consumption setting, a sampling rate setting, a compression rate setting, and a communication setting.

12. The implantable medical device according to claim 1, wherein the processing module is configured to initiate said second mode of operation based on a detection of at least one predefined event.

13. A method for operating an implantable medical device for sensing electrocardiogram signals, comprising:
sensing electrocardiogram signals using an arrangement of electrode poles of the implantable medical device;
processing, using a processing module of the implantable medical device, electrocardiogram signals received via said arrangement of electrode poles;
identifying, by the processing module in a first mode of operation, a predefined event based on sensed electrocardiogram signals and recording a snapshot signal representation of electrocardiogram signals over a first duration of time; and
recording, by the processing module in a second mode of operation, a continuous signal representation of electrocardiogram signals over a second duration of time larger than said first duration of time.
